(19) United States
(12) Patent Application Publication
(10) Pub. No.: US 2007/0038190 A1
(43) Pub. Date: Feb. 15, 2007

(54) Hygiene system

(76) Inventors: Martin Gerard Grimer, Thame (GB); Mark Edward Laws, Woodbridge (GB)

Correspondence Address: KIMBERLY-CLARK WORLDWIDE, INC., 401 NORTH LAKE STREET, NEENAH, WI, 54956, US

(21) Appl. No.: 11/192,210

(22) Filed: Jul. 28, 2005

(51) Int. Cl. A61M 35/00

(52) U.S. Cl. 604/292

(57) ABSTRACT

A product has one or more morphics associated therewith. The morphics include an alphabetic base letter and one or more visual characteristics associated with an object. The object name starts with the base letter. The product name may start with the alphabetic base letter. The objects may be animals. The morphics may have proper names starting with the alphabetic base letter. Two or more of the products may form an array wherein the morphics are uniquely associated with different products.

### COPYRIGHT NOTICE

A portion of the disclosure of this patent document contains material which may be subject to copyright and/or trademark protection. The copyright and trademark owner has no objection to the facsimile reproduction by anyone of the patent document or the patent disclosure, as it appears in the Patent and Trademark Office patent file or records, but otherwise reserves all copyright and trademark rights whatsoever.

### BACKGROUND OF THE INVENTION

Personal hygienic activities such as bathing, hair maintenance, skin care, body cleansing and toileting are a necessary part of life. As such, various consumer products such as shampoo, bath wash, soap, mitts, wipes and the like are available for use by adults and children. However, children may not appreciate the necessity of personal hygiene and may find the associated activities to be unpleasant. This is a cause of struggle for many children and for the people caring for them. In response, various consumer products have included bright colors or characters on the packaging in an effort to make personal hygiene easier on kids and caregivers.

However, there is still a need for products that make hygiene fun, encourage interaction between parents and kids, and help children learn to care for themselves.

### SUMMARY OF THE INVENTION

In response to these needs, the inventors have acted on the observation that when a child learns through play, everything comes more naturally. As such, a range of products directed towards children makes learning to keep clean a lot more fun and creates a fun and interactive hygienic experience.

In one aspect a product has one or more morphics associated with it. The morphics include an alphabetic base letter combined with one or more visual characteristics associated with an object. The object has an object name starting with the alphabetic base letter.

In various embodiments, the product may have a product name starting with the alphabetic base letter and/or the object may be an animal and/or the morphics may include a graphic representing a proper name starting with the alphabetic base letter.

In various embodiments, the product may be contained within a package having at least one first morphic printed directly on the package or on a label adhered to the package. In some embodiments, the product contained within the package may include at least one second morphic. The first morphic and the second morphics may be the same.

In various embodiments, the product may be shampoo, detangler, bath wash, mitts, body wash, wipes, flushable wipes, cleansing cloths or hand soap.

In another aspect, an array of products includes at least one first product and at least one second product. The first and second products are different. The first product has a first product name. The first product has a first morphic associated therewith. The first morphic includes a first alphabetic base letter combined with one or more visual characteristics associated with a first object. The first object has a first object name. The first product name and the first object name start with the first alphabetic base letter. The second product has a second product name. The second product has a second morphic associated therewith. The second morphic includes a second alphabetic base letter combined with one or more visual characteristics associated with a second object. The second object has a second object name. The second product name and the second object name start with the second alphabetic base letter. The first morphic is uniquely associated with the first product. The second morphic is uniquely associated with the second product.

In various embodiments, the first object is a first animal and the second object is a second animal. In various embodiments, the animals are a snake, mule, bison, dolphin, flamingo, crab, lion, or hippopotamus.

In various embodiments, the first morphic includes a graphic representing a proper name starting with the first alphabetic base letter and the second morphic includes a graphic representing a proper name starting with the second alphabetic base letter.

In various embodiments, a first package contains the first product and a second package contains the second product. The first package may have the first morphic printed thereon. The second package may have the second morphic printed thereon. The morphics may also be printed on labels and adhered to the first and/or second packages.

In various embodiments, the products may include shampoo disposed within a shampoo container displaying a snake alphamal, mitts disposed within a mitts container displaying a mule alphamal, body wash disposed within a body wash container displaying a bison alphamal, detangler disposed within a detangler container displaying a dolphin alphamal, flushable wipes disposed within a flushable wipes container displaying a flamingo alphamal, cleansing cloths disposed within a cleansing cloth container displaying a crab alphamal, lotion disposed within a lotion container displaying a lion alphamal or hand soap disposed within a hand soap container displaying a hippo alphamal.

In another aspect, a hygiene product includes one or more alphamals associated therewith. The alphamals include an alphabetic base letter combined with one or more visual characteristics associated with an animal. The animals have an animal name starting with the alphabetic base letter.

In various embodiments, the hygiene product has a product name starting with the alphabetic base letter.

In various embodiments, the alphamal includes a graphic representing a proper name starting with the alphabetic base letter.

In various embodiments, the hygiene product is contained within a package and at least one first alphamal is printed on the package or on a label adhered to the package. In various embodiments, a second alphamal is printed on the hygiene product and the first alphamal and the second alphamal are the same.

In various embodiments, the hygiene product may be shampoo, detangler, bath wash, mitts, body wash, wipes, flushable wipes, cleansing cloths or hand soap.

In various embodiments, the animal is a snake, mule, bison, dolphin, flamingo, crab, or hippopotamus.

In various embodiments, the alphamal has the proper name Sammy, Molly, Billy, Daphne, Freddy, Carly or Henry.

### BRIEF DESCRIPTION OF THE DRAWINGS

FIG. 1 representatively illustrates a morphic.

FIG. 2 representatively illustrates a morphic associated with a hygiene product.

FIG. 3 representatively illustrates an array of hygiene products and the associated morphics.

### DETAILED DESCRIPTION OF THE DRAWINGS

The features of the present invention will be described in connection with hygiene products adapted for infants, toddlers and young children. However, the features of the present invention may also be applicable to other products such as clothing, toys, food products, and the like.

As used herein, a “morphic” is a single graphic formed by the combination of an upper or lower case alphabetic base letter and one or more visual characteristics associated with an object having an object name, wherein the object name starts with the base letter. For example, a tree morphic is the combination of the alphabetic letter “t” and one or more of the visual characteristics associated with a tree, such as leaves and/or branches, merged together to form a graphic. Some, all, or none of the morphics may have eyes or eyeballs.

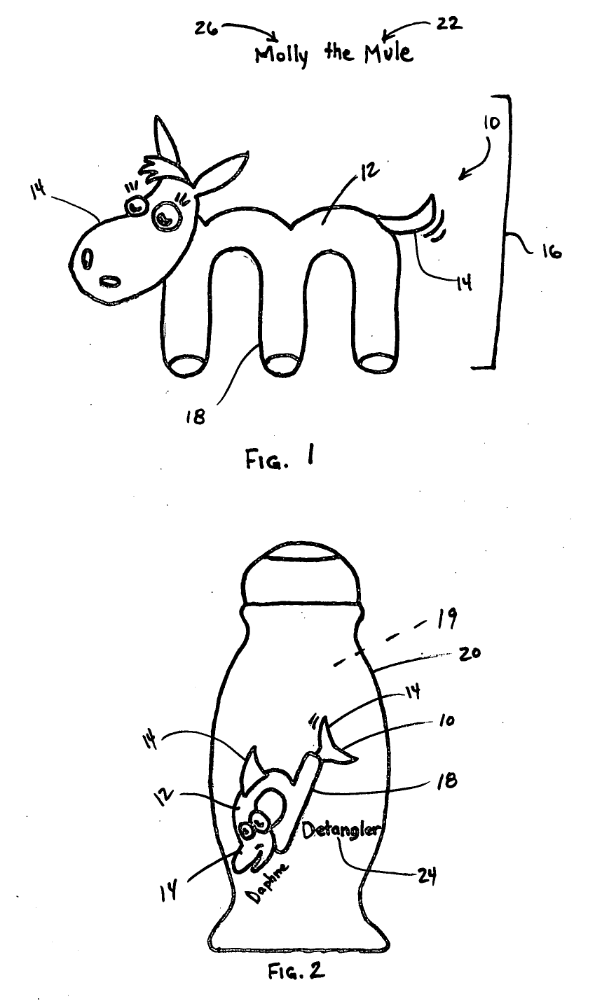

Referring to FIG. 1, a representative morphic is illustrated generally at 10. The morphic 10 comprises a lower case alphabetic base letter 12 combined with visual characteristics 14 associated with an object to create a graphic 16. The object has an object name 22 and the object name 22 starts with the base letter 12. The object may also have a proper name 26. In this example, the alphabetic base letter 12 is an “m”, the object is a mule, the object name 22 is “mule”, and the proper name 26 is “Molly”. The visual characteristics 14 associated with the object are a mule's head and tale. Because the object is an animal, this morphic 10 is an alphamal 18, specifically a mule alphamal 18.

As used herein, an “alphamal” is a single graphic formed by the combination of an upper or lower case alphabetic base letter and one or more visual characteristics associated with an animal having an animal name, wherein the animal name starts with the base letter.

As another example, a snake alphamal is the combination of the base letter “s” and one or more of the visual characteristics associated with snakes, such as, for example, a thin forked tongue.

As another example, a bison alphamal is the combination of the base letter “b” and one or more of the visual characteristics associated with bison, such as, for example, horns, legs and hair under the mouth.

As another example, a flamingo alphamal is the combination of the base letter “f” and one or more of the visual characteristics associated with flamingos, such as, for example, the color pink, wings and a beak.

As another example, a crab alphamal is the combination of the base letter “c” and one or more of the visual characteristics associated with crabs, such as, for example, claws, legs, and eyes on stalks.

As another example, a dolphin alphamal is the combination of the base letter “d” and one or more of the visual characteristics associated with dolphins, such as, for example, a dolphin face, a dorsal fin and a tale.

As another example, a hippo alphamal is the combination of the base letter “h” and one or more of the visual characteristics associated with hippopotamus, such as, for example, a large belly, tail, small rounded ears, large round face and jaw and large teeth.

In various embodiments, the one or more visual characteristics may be realistic portrayals of the given object or may be cartoonish portrayals of the given object or combinations thereof. For example, in some embodiments, the visual characteristics may be cartoonish portrayals of characteristics associated with animals.

In various embodiments, the morphics may have proper names, such as, for example, Sammy, Molly, Billy, Daphne, Freddy, Carly, Henry, Lenny, and the like. The proper names may start with a letter, and the letter may be the same as the base letter that comprises the morphic. For example, a morphic having the base letter “L” may have the proper name “Lenny”. In another example, a morphic having the base letter “c” may have the proper name “Carly”.

In various embodiments, the object name and/or the proper name may be graphically represented on or near the morphic associated therewith. Additionally or alternatively the product name may be graphically represented on or near the product associated therewith.

In various embodiments, one or more morphics may be associated with one or more products. For example, morphics may be associated with products by being printed on the products or on labels attached to the products. For example, morphics may be printed on mitts, cleansing cloths, wipes and the like and combinations thereof. In various embodiments, one or more morphics may be associated with products by being associated with packages containing one or more of the products. For example, morphics may be printed on packages, or on labels attached to packages, containing shampoo, body wash, detangler, soap, lotion, ointment, and the like, and combinations thereof. In some embodiments, morphics may be included on both the package containing the product and the product contained therein. For example, morphics may be printed on mitts and may be printed on the package that contains the mitts.

In various embodiments, the morphic may be coordinated with the product with which it is associated. As used herein, a morphic is “coordinated with the product” when the alphabetic base letter of the morphic is the first letter of the product name. For example, a morphic including the alphabetic base letter “s” located on a package containing “shampoo” is coordinated with the shampoo product. Similarly, a morphic including the alphabetic base letter “b” located on a package containing “bath wash” is coordinated with the bath wash product.

In various embodiments, the morphic and product may be further coordinated. The product may have a product name. The morphic may have a proper name. The alphabetic base letter of the morphic and/or the first letter of the object name and/or the first letter of the proper name and/or the first letter of the product name may be the same.

For example, a morphic may be an alphamal comprising an alphabetic base letter “h” combined with one or more visual characteristics associated with a hippopotamus to form a hippo alphamal. The hippopotamus has an object name, “hippopotamus”. The alphamal may have a proper name, such as, for example, “Henry”. The alphamal may be included on a container of handsoap having a product name “handsoap”. The base letter “h”, the object name “hippopotamus”, the proper name “Henry” and the product name “handsoap” all start with the letter “h”.

In another example, a morphic may be an alphamal comprising an alphabetic base letter “b” combined with one or more visual characteristics of a bison to form a bison alphamal. The bison has an object name, “bison”. The alphamal may have a proper name, such as, for example, “Billy”. The alphamal may be included on a package containing bath wash having a product name “bath wash”. The base letter “b”, the object name “bison”, the proper name “Billy” and the product name “bath wash” all start with the letter “b”.

In another example, a morphic may be an alphamal comprising an alphabetic base letter “f” combined with one or more visual characteristics associated with a flamingo to form a flamingo alphamal. The flamingo has an object name, “flamingo”. The alphamal may have a proper name, such as, for example, “Freddy”. The alphamal may be included on a package containing flushable wipes having a product name “flushable wipes”. The base letter “f”, the object name “flamingo”, the proper name “Freddy” and the product name “flushable wipes” all start with the letter “f”.

In another example, a morphic may be an alphamal comprising an alphabetic base letter “c” combined with one or more visual characteristics associated with a crab to form a crab alphamal. The crab has an object name, “crab”. The alphamal may have a proper name, such as, for example, “Carly”. The alphamal may be included on a package containing cleansing cloths having a product name “cleansing cloths”. The base letter “c”, the object name “crab”, the proper name “Carly” and the product name “cleansing cloths” all start with the letter “c”.

In another example, a morphic may comprise an alphabetic base letter “t” combined with one or more visual characteristics associated with a tree to form a tree morphic. The tree has an object name, “tree”. The morphic may have a proper name, such as, for example, “Tony”. The morphic may be included on a package containing taco chips having a product name “taco chips”. The base letter “t”, the object name “tree”, the proper name “Tony” and the product name “taco chips” all start with the letter “t”.

In various embodiments, the base letter and/or the object name and/or the proper name and/or the product name may be in a language other than English, such as, for example, Spanish, French, German, Japanese, Korean, and the like.

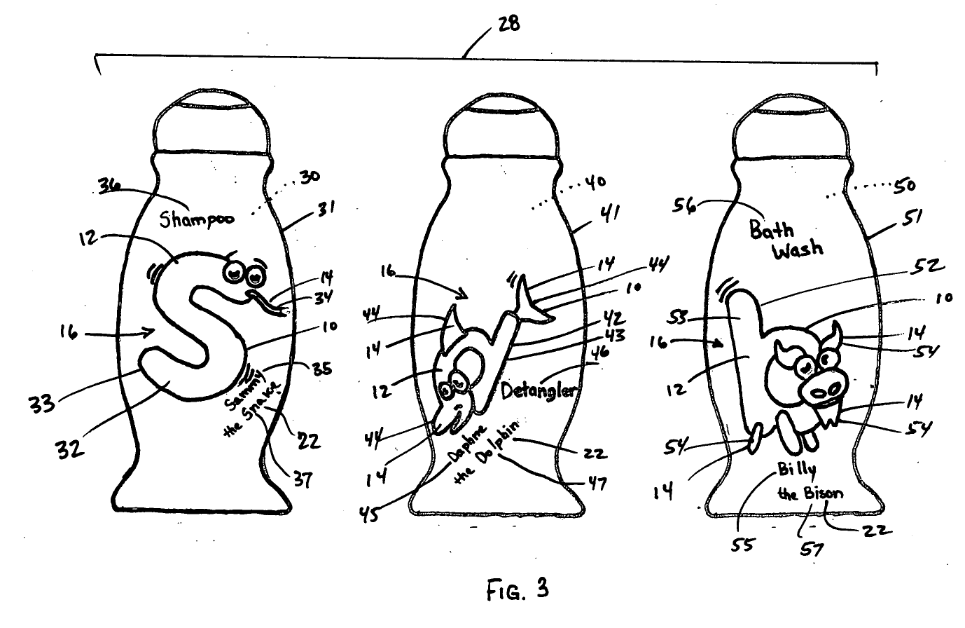

Referring now to FIG. 2, a morphic 10, specifically an alphamal 18, is associated with a product 19 by being associated with a package 20 containing the product 19. The product 19 is detangler and the product name 24 is “detangler”. As used herein, the term “package” refers to a covering wrapper or container. The association of the alphamal 18 and the package 20 may be made by any suitable means, such as, for example, printing on the package, printing on a label attached to the package, and the like and combinations thereof.

The alphamal 18 includes an alphabetic base letter 12, “d”, combined with visual characteristics 14 associated with an object. The object is a dolphin and has an object name 22, “dolphin”. The first letter of the object name 22 and the base letter 12 both start with the letter “d”. The visual characteristics 14 are a dolphin head, dorsal fin and tail. The dolphin alphamal 18 is coordinated with the detangler as both the base letter 12 of the alphamal 18 and the first letter of the product name 24, “detangler”, start with the letter “d.”

Additionally, the alphamal 18 has a proper name 26, “Daphne”. The base letter 12, “d”, the object name 22, “dolphin”, the product name 24, “detangler”, and the proper name 26, “Daphne”, all start with the letter “d”.

In various embodiments, at least one first consumer product may have a first morphic associated therewith and at least one second consumer product may have a second morphic associated therewith to define an array of consumer products. Each type of consumer product in the array, for example, shampoo, handsoap, detangler and flushable wipes, may have a morphic uniquely associated therewith.

As used herein, the term “uniquely associated” refers to a specific morphic being associated with a specific type of product and no other type of product. For example, a snake alphamal may be associated only with shampoo, a bison alphamal may be associated only with bath wash, a flamingo alphamal may be associated only with flushable wipes, a crab alphamal may be associated only with cleansing cloths, a dolphin alphamal may be associated only with detangler, a hippo alphamal may be associated only with hand soap, a mule alphamal may be associated only with mitts, and the like.

Each array of products may have a common theme and/or style and the morphics may conform to the theme and/or style such that a given product may be associated with an array of products based on the morphic associated therewith. Exemplary themes, may include, animals, sports, vegetables, professions and the like. The morphics may conform to the themes by comprising objects associated with the theme. For example, an array of consumer products may have the theme “animals” and the morphics may include objects that are “animals”. In another example, the array of consumer products may have the theme “sports” and the morphics may include objects that are associated with sports, such as, for example, footballs, golf balls, tennis shoes, helmets, goal posts, soccer balls and the like.

Referring now to FIG. 3, an exemplary array of products is shown generally at 28. The array 28 includes three consumer products, shampoo 30, detangler 40 and body wash 50. The consumer products 30, 40 and 50 are contained within packages 31, 41 and 51 respectively. Each package 31, 41 and 51 has a morphic 10 associated therewith. The morphics 10 comprise an alphabetic base letter 12 combined with one or more visual characteristics 14 associated with an object to create a graphic 16. The object has an object name 22 and the object name 22 starts with the base letter 12.

The first consumer product in the exemplary array 28, shampoo 30, is contained within a shampoo package 31 and has a product name 36, “shampoo”. The shampoo package 31 has a snake alphamal 32 associated therewith. The snake alphamal 32 comprises an alphabetic base letter 33, “s”. The base letter 33, “s”, is combined with a visual characteristic 34 to form a graphic 16. The visual characteristic 34 is associated with an object having an object name 37. The object is a snake and the object name 37 is “snake”. The visual characteristic 34 is a thin forked tongue as is commonly associated with snakes. The alphamal 32 has a proper name 35, “Sammy”. The base letter 33, the object name 37, the product name 36 and the proper name 35 all start with the letter “s”. This association helps the child connect the letter “s” with an animal, a product and a proper name. Therefore, even children that cannot yet read may learn and remember which package 31 contains the shampoo 30.

The second consumer product in the exemplary array 28, detangler 40, is contained within a detangler package 41 and has a product name 46, “detangler”. The detangler package 41 has a dolphin alphamal 42 associated therewith. The dolphin alphamal 42 comprises an alphabetic base letter 43, “d”. The base letter 43, “d”, is combined with visual characteristics 44 to form a graphic 16. The visual characteristics 44 are associated with an object having an object name 47. The object is a dolphin and the object name 47 is “dolphin”. The visual characteristics 44 are a dolphin head, dorsal fin and tail as are commonly associated with dolphins. The alphamal 42 has a proper name 45, “Daphne”. The base letter 43, the object name 47, the product name 46 and the proper name 45 all start with the letter “d”. This association helps the child connect the letter “d” with an animal, a product and a proper name. Therefore, even children that cannot yet read may learn and remember which package 41 contains the detangler 40.

The third consumer product in the exemplary array 28, bath wash 50, is contained within a bath wash package 51 and has a product name 56, “bath wash”. The bath wash package 51 has a bison alphamal 52 associated therewith. The bison alphamal 52 comprises an alphabetic base letter 53, “b”. The base letter 53, “b”, is combined with visual characteristics 54 to form a graphic. The visual characteristics 54 are associated with an object having an object name 57. The object is a bison and the object name 57 is “bison”. The visual characteristics 54 are horns, legs and hair below the chin as are commonly associated with bison. The alphamal 52 has a proper name 55, “Billy”. The base letter 53, the object name 57, the product name 56 and the proper name 55 all start with the letter “b”. This association helps the child connect the letter “b” with a particular animal, product and proper name. Therefore, even children that cannot yet read may learn and remember which package 51 contains the bath wash 50.

The products 30, 40 and 50 comprising the array 28 have different morphics 10 uniquely associated with each. Each morphic 10 has the common theme of being formed by an alphabetic base letter and one or more characteristics associated with an animal. The animal theme creates an association between products within the array 28. A child familiar with Sammy the Snake Alphamal Shampoo may be more likely to recognize and use Daphne the Dolphin Alphamal Detangler because of the thematic association.

Additionally, the themes may be expanded through the addition of books, toys, videos and the like that feature the morphics. For example, a Billy the bison alphamal toy suitable for use in the bath tub may increase the enjoyment of using Billy the Bison body wash. In another example, an array of products comprising morphics may have an animal theme and a book, video or the like and may feature “alphamal island” where all the alphamals gather to play and live. A parent or caregiver may continue the interaction enjoyed during bath time by reading a book about the alphamals to the child or watching the alphamals on television with the child.

While not wishing to be bound by theory, it is believed that creating an interactive learning experience through the use of morphics associated with consumer products and linked together by themes can make hygienic activities fun for kids and caregivers. Kids have fun learning their letters and associating the letters with animals, names and products. Making hygiene fun can alleviate the common struggle with kids that don't want to participate in hygienic activities such as bathing, hair maintenance, cleansing, toileting and the like.

Additionally, it is believed that kids associate the various morphics with the various products and learn to identify the type of product by the morphic.

This association may help a child develop independence using toiletries by learning how to identify and properly use various products before they can read.

While the invention has been described in detail with respect to specific embodiments thereof, it will be appreciated that those skilled in the art, upon attaining an understanding of the foregoing will readily conceive of alterations to, variations of and equivalents to these embodiments. Accordingly, the scope of the present invention should be assessed as that of the appended claims and any equivalents thereto.

1. A product comprising one or more morphics associated therewith.

2. The product of claim 1 wherein the one or more morphics are coordinated with the product.

3. The product of claim 2 wherein the morphic is an alphamal.

4. The product of claim 3 wherein the morphic includes a graphic representing a proper name starting with the alphabetic base letter.

5. A package containing at least the product of claim 1, wherein at least one first morphic is printed on the package or on a label adhered to the package.

6. The package of claim 5 wherein at least one second morphic is printed on the product and wherein the first morphic and the second morphic are the same.

7. The product of claim 2 wherein the product is selected from the group consisting of, shampoo, detangler, bath wash, mitts, body wash, wipes, flushable wipes, cleansing cloths and hand soap.

8. An array of products comprising,
at least one first product having a first product name, the first product having a first morphic associated therewith, the first morphic comprising a first alphabetic base letter combined with one or more visual characteristics associated with a first object, the first object having a first object name, the first product name and the first object name starting with the first alphabetic base letter,
at least one second product having a second product name, the second product having a second morphic associated therewith, the second morphic comprising a second alphabetic base letter combined with one or more visual characteristics associated with a second object, the second object having a second object name, the second product name and the second object name starting with the second alphabetic base letter,
wherein the first morphic is uniquely associated with the first product, the second morphic is uniquely associated with the second product and the first and second products are different.

9. The array of claim 8 wherein the first object is a first animal and the second object is a second animal.

10. The array of claim 9 wherein the first animal and the second animal are selected from the group consisting of snake, mule, bison, dolphin, flamingo, crab, lion, and hippopotamus.

11. The array of claim 9 wherein the first morphic includes a graphic representing a proper name starting with the first alphabetic base letter and the second morphic includes a graphic representing a proper name starting with the second alphabetic base letter.

12. The array of claim 8 further comprising a first package and a second package wherein the first product is contained within the first package and the second product is contained within the second package, wherein the first morphic is a graphic printed on the first package or on a label adhered to the first package and the second morphic is a graphic printed on the second package or on a label adhered to the second package.

13. The array of claim 12 wherein the first product and the second product are selected from the group consisting of shampoo disposed within a shampoo container displaying a snake alphamal, mitts disposed within a mitts container displaying a mule alphamal, body wash disposed within a body wash container displaying a bison alphamal, detangler disposed within a detangler container displaying a dolphin alphamal, flushable wipes disposed within a flushable wipes container displaying a flamingo alphamal, cleansing cloths disposed within a cleansing cloth container displaying a crab alphamal, lotion disposed within a lotion container displaying a lion alphamal and hand soap disposed within a hand soap container displaying a hippo alphamal.

14. A hygiene product comprising,
at least one alphamal associated therewith, the alphamal comprising, an alphabetic base letter combined with one or more visual characteristics associated with an animal, the hygiene product having a product name starting with the alphabetic base letter.

15. The hygiene product of claim 14 wherein the alphamal includes a graphic representing a proper name starting with the alphabetic base letter.

16. A package containing at least the product of claim 14, wherein at least one first alphamal is printed on the package or on a label adhered to the package.

17. The package of claim 16 wherein at least one second alphamal is printed on the hygiene product and the first alphamal and the second alphamal are the same.

18. The hygiene product of claim 15 wherein the hygiene product is selected from the group consisting of, shampoo, detangler, bath wash, mitts, body wash, wipes, flushable wipes, cleansing cloths and hand soap.

19. The hygiene product of claim 18 wherein the animal is selected from the group consisting of snakes, mules, bison, dolphin, flamingo, crab, and hippopotamus.

20. The hygiene product of claim 19 wherein the proper name is selected from the group consisting of Sammy, Molly, Billy, Daphne, Freddy, Carly and Henry.
